(19) United States
(12) Patent Application Publication
(10) Pub. No.: US 2022/0240848 A1
(43) Pub. Date: Aug. 4, 2022

(54) Systems and Methods for Risk Based Insulin Delivery Conversion

(71) Applicant: Dexcom, Inc., San Diego, CA (US)

(72) Inventor: Stephen D. Patek, San Diego, CA (US)

(73) Assignee: Dexcom, Inc., San Diego, CA (US)

(21) Appl. No.: 17/592,137

(22) Filed: Feb. 3, 2022

Related U.S. Application Data
(63) Continuation of application No. 17/592,071, filed on Feb. 3, 2022.
(60) Provisional application No. 63/145,224, filed on Feb. 3, 2021.

(51) Int. Cl. A61B 5/00; G16H 20/17; A61M 5/172

(52) U.S. Cl. CPC A61B 5/4839; G16H 20/17; A61M 2230/201; A61B 5/7275; A61M 5/1723

(57) ABSTRACT

Systems and methods are provided for managing hyperglycemia and hypoglycemia by reconciling incoming data to provide safe and reliable control to range using automatic bolus determination wherein the rate of insulin delivery is dependent on the level of hyperglycemic risk or hypoglycemic risk. Additionally, some implementations are directed to converting insulin delivery into a rate based on glycemic risk.

### CROSS-REFERENCE TO RELATED APPLICATIONS

This application is a Continuation of and claims the benefit of priority to U.S. Non-Provisional patent application Ser. No. 17/592,071, filed on Feb. 3, 2022 which claims the benefit of priority to U.S. Provisional Patent Application No. 63/145,224, filed on Feb. 3, 2021, entitled “SYSTEMS AND METHODS FOR RISK BASED INSULIN DELIVERY CONVERSION,” the contents of which are hereby incorporated by reference in their entirety.

### BACKGROUND

With the growing adoption of continuous glucose monitoring (CGM) and connected devices, the availability and reliability of glucose time-series data has increased in recent years. However, despite the availability of reliable glucose data, accurate tracking of insulin and meal data and optimized and effective timing of meal time insulin bolusing continues to be problematic for many people with diabetes resulting in poor glucose control.

Prior diabetes management algorithms have developed iteratively over time and include numerous modules that may overlap or even conflict in function in an effort to provide flexibility to the various user considerations and interactions.

It is with respect to these and other considerations that the various aspects and embodiments of the present disclosure are presented.

### SUMMARY

Systems and methods are provided for managing hyperglycemia and hypoglycemia by reconciling incoming data to provide safe and reliable control to range using automatic bolus determination wherein the rate of insulin delivery is dependent on the level of hyperglycemic risk or hypoglycemic risk. Additionally, some implementations are directed to converting insulin delivery into a rate based on glycemic risk.

In an implementation, a risk based insulin delivery rate converter comprises: a comparator that is configured to receive insulin data and glucose data, and comprises a model agreement assessor configured to identify a discrepancy between differently derived estimations of metabolic data and behavioral data derived from the insulin data and the glucose data, by quantifying the degree to which recent blood glucose measurements are inconsistent with recent insulin; a glycemic risk assessor configured to quantify the risk of at least one of current or future hyperglycemia or hypoglycemia based on the glucose data; and an insulin delivery supervisor configured to modulate insulin delivery rates based on data from the comparator and the glycemic risk assessor.

In an implementation, a risk based insulin delivery rate conversion method comprises: receiving insulin data and glucose data at a comparator; identifying a discrepancy between differently derived estimations of metabolic data and behavioral data derived from the insulin data and the glucose data, using a model agreement assessor of the comparator, by quantifying the degree to which recent blood glucose measurements are inconsistent with recent insulin; quantifying the risk of at least one of current or future hyperglycemia or hypoglycemia based on the glucose data, using a glycemic risk assessor; and modulating insulin delivery rates based on data from the comparator and the glycemic risk assessor, using an insulin delivery supervisor.

In an implementation, a system comprises: at least one processor; and a non-transitory computer readable medium comprising instructions that, when executed by the at least one processor, cause the system to: receive insulin data and glucose data at a comparator; identify a discrepancy between differently derived estimations of metabolic data and behavioral data derived from the insulin data and the glucose data, using a model agreement assessor of the comparator, by quantifying the degree to which recent blood glucose measurements are inconsistent with recent insulin; quantify the risk of at least one of current or future hyperglycemia or hypoglycemia based on the glucose data, using a glycemic risk assessor; and modulate insulin delivery rates based on data from the comparator and the glycemic risk assessor, using an insulin delivery supervisor.

In an implementation, a risk based insulin delivery rate converter comprises: a comparator that is configured to receive insulin data and glucose data, and comprises a model agreement assessor configured to identify a discrepancy between differently derived estimations of metabolic data and behavioral data derived from the insulin data and the glucose data, by quantifying the degree to which recent blood glucose measurements are inconsistent with recent insulin; a glycemic risk assessor configured to quantify the risk of at least one of current or future hyperglycemia or hypoglycemia based on the glucose data; an insulin delivery supervisor configured to modulate insulin delivery rates based on data from the comparator and the glycemic risk assessor; and a reference insulin rate (RIR) updater configured to determine a RIR, wherein the RIR is an internal reference for insulin that would achieve equilibrium.

In an implementation, a risk based insulin delivery rate conversion method comprises: receiving insulin data and glucose data at a comparator; identifying a discrepancy between differently derived estimations of metabolic data and behavioral data derived from the insulin data and the glucose data, using a model agreement assessor of the comparator, by quantifying the degree to which recent blood glucose measurements are inconsistent with recent insulin; quantifying the risk of at least one of current or future hyperglycemia or hypoglycemia based on the glucose data, using a glycemic risk assessor; modulating insulin delivery rates based on data from the comparator and the glycemic risk assessor, using an insulin delivery supervisor; and determining a reference insulin rate (RIR) using a RIR updater, wherein the RIR is an internal reference for insulin that would achieve equilibrium.

In an implementation, a system comprises: at least one processor; and a non-transitory computer readable medium comprising instructions that, when executed by the at least one processor, cause the system to: receive insulin data and glucose data at a comparator; identify a discrepancy between differently derived estimations of metabolic data and behavioral data derived from the insulin data and the glucose data, using a model agreement assessor of the comparator, by quantifying the degree to which recent blood glucose measurements are inconsistent with recent insulin; quantify the risk of at least one of current or future hyperglycemia or hypoglycemia based on the glucose data, using a glycemic risk assessor; modulate insulin delivery rates based on data from the comparator and the glycemic risk assessor, using an insulin delivery supervisor; and determine a reference insulin rate (RIR) using a RIR updater, wherein the RIR is an internal reference for insulin that would achieve equilibrium.

In an implementation, a method comprises: receiving a plurality of inputs at a comparator; identifying discrepancies between differently derived estimations of metabolic data and behavioral data derived from inputs; quantifying the risk of current or future hyperglycemia or hypoglycemia based on the glucose data, using a glycemic risk assessor; and modulating insulin delivery rates, using an insulin delivery supervisor, based on data from the comparator and from the glycemic risk assessor.

In an implementation, a system comprises: a comparator configured to receive a plurality of inputs and identify discrepancies between differently derived estimations of metabolic data and behavioral data derived from the inputs; a glycemic risk assessor configured to quantify the risk of current or future hyperglycemia or hypoglycemia based on the glucose data; and an insulin delivery supervisor configured to modulate insulin delivery rates based on data from the comparator and from the glycemic risk assessor.

In an implementation, a system comprises: at least one processor; and a non-transitory computer readable medium comprising instructions that, when executed by the at least one processor, cause the system to: receive a plurality of inputs at a comparator; identify discrepancies between differently derived estimations of metabolic data and behavioral data derived from inputs; quantify the risk of current or future hyperglycemia or hypoglycemia based on the glucose data, using a glycemic risk assessor; and modulate insulin delivery rates, using an insulin delivery supervisor, based on data from the comparator and from the glycemic risk assessor.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is a high level functional block diagram of an embodiment of the invention;

FIG. 2 is a block diagram of an implementation of a risk based insulin delivery rate converter;

FIG. 3 is a flow diagram of an implementation of a method of risk based insulin delivery rate conversion;

FIG. 4 is a block diagram of an implementation of a comparator for use with risk based insulin delivery rate conversion;

FIG. 5 is a flow diagram of an implementation of a method of comparison for use with risk based insulin delivery rate conversion;

FIG. 6 is a block diagram of an implementation of a glycemic risk assessor for use with risk based insulin delivery rate conversion;

FIG. 7 is a flow diagram of an implementation of a method of glycemic risk assessment for use with risk based insulin delivery rate conversion;

FIG. 8 is a block diagram of an implementation of an insulin delivery supervisor for use with risk based insulin delivery rate conversion;

FIG. 9 is a flow diagram of an implementation of a method of insulin delivery supervision for use with risk based insulin delivery rate conversion; and

FIG. 10 shows an exemplary computing environment in which example embodiments and aspects may be implemented.

### DETAILED DESCRIPTION

The claimed subject matter is described with reference to the drawings, wherein like reference numerals are used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

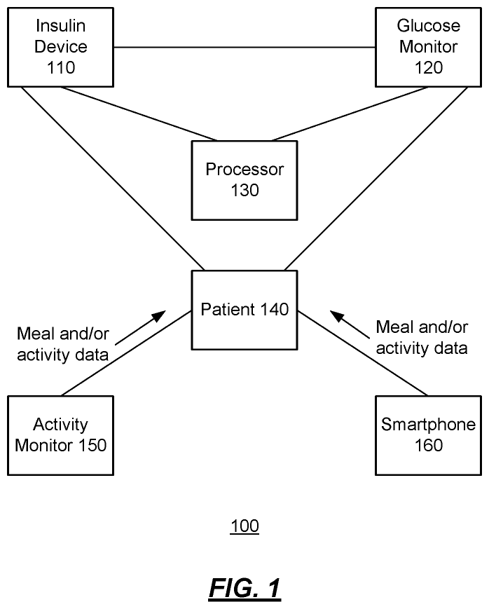

FIG. 1 is a high level functional block diagram 100 of an embodiment of the invention. A processor 130 communicates with an insulin device 110 and a glucose monitor 120. The insulin device 110 and the glucose monitor 120 communicate with a patient 140 to deliver insulin to the patient 140 and monitor glucose levels of the patient 140, respectively. The processor 130 is configured to perform the calculations and other operations and functions described further herein. The insulin device 110 and the glucose monitor 120 may be implemented as separate devices or as a single device, within a single device, or across multiple devices. The processor 130 can be implemented locally in the insulin device 110, the glucose monitor 120, or as a standalone device (or in any combination of two or more of the insulin device 110, the glucose monitor 120, or a standalone device). The processor 130 or a portion of the system shown can be located remotely such as within a server or a cloud-based system.

Examples of insulin devices, such as the insulin device 110, include insulin syringes, external pumps, and patch pumps that deliver insulin to a patient, typically into the subcutaneous tissue. Insulin devices 110 also includes devices that deliver insulin by different means, such as insulin inhalers, insulin jet injectors, intravenous infusion pumps, and implantable insulin pumps. In some embodiments, a patient will use two or more insulin delivery devices in combination, for example injecting long-acting insulin with a syringe and using inhaled insulin before meals. In other embodiments, these devices can deliver other drugs that help control glucose levels such as glucagon, pramlintide, or glucose-like peptide-1 (GLP-1).

Examples of a glucose monitor, such as the glucose monitor 120, include continuous glucose monitors that record glucose values at regular intervals, e.g., 1, 5, or 10 minutes, etc. These continuous glucose monitors can use, for example, electrochemical or optical sensors that are inserted transcutaneously, wholly implanted, or measure tissue noninvasively. Examples of a glucose monitor, such as the glucose monitor 120, also include devices that draw blood or other fluids periodically to measure glucose, such as intravenous blood glucose monitors, microperfusion sampling, or periodic finger sticks. In some embodiments, the glucose readings are provided in near realtime. In other embodiments, the glucose reading determined by the glucose monitor can be stored on the glucose monitor itself for subsequent retrieval.

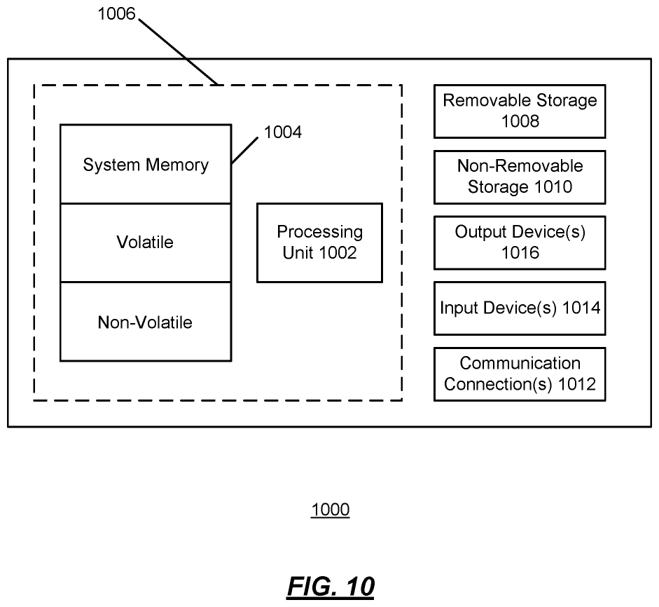

The insulin device 110, the glucose monitor 120, and the processor 130 may be implemented using a variety of computing devices such as smartphones, desktop computers, laptop computers, and tablets. Other types of computing devices may be supported. A suitable computing device is illustrated in FIG. 10 as the computing device 1000 and cloud-based applications.

The insulin device 110, the glucose monitor 120, and the processor 130 may be in communication through a network. The network may be a variety of network types including the public switched telephone network (PSTN), a cellular telephone network, and a packet switched network (e.g., the Internet). Although only one insulin device 110, one glucose monitor 120, and one processor 130 are shown in FIG. 1, there is no limit to the number of insulin devices, glucose monitors, and processors that may be supported. An activity monitor 150 and/or a smartphone 160 may also be used to collect meal and/or activity data from or pertaining to the patient 140, and provide the meal and/or activity data to the processor 130.

The processor 130 may execute an operating system and one or more applications. The operating system may control which applications are executed by the insulin device 110 and/or the glucose monitor 120, as well as control how the applications interact with one or more sensors, services, or other resources of the insulin device 110 and/or the glucose monitor 120.

The processor 130 receives data from the insulin device 110 and the glucose monitor 120, as well as from the patient 140 in some implementations, and may be configured and/or used to perform one or more of the calculations, operations, and/or functions described further herein.

Risk based insulin delivery conversion as contemplated and described herein is applicable to any conventional diabetes management platform designed to determine and/or deliver insulin delivery rates for a patient. Applicable embodiments include but are not limited to: conventional fully manual open loop therapy, decision support therapy, control to range automated insulin delivery (AID), control to target AID, model predictive control (MPC), linear quadratic Gaussian (LQG), proportional integral derivative (PID), or the like. In some implementations, as described further herein, an insulin delivery supervisor (e.g., the insulin delivery supervisor 245 described further herein) modulates insulin delivery rates based on discrepancies in expected versus actual metabolic states and hyperglycemic risk levels.

Moreover, according to some implementations, an artificial pancreas (AP) algorithm is provided that manages hyperglycemia by reconciling incoming data to provide safe and reliable control to range using automatic bolus determination wherein the rate of insulin delivery is dependent on the level of hyperglycemic risk. Further embodiments may be implemented to address hypoglycemic risk. Additionally, some implementations are directed to converting insulin delivery into a rate based on glycemic risk.

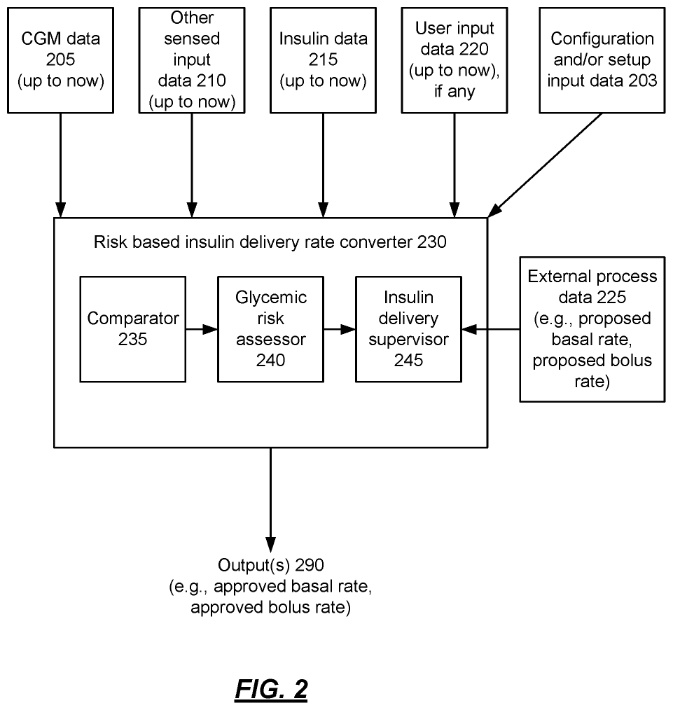

FIG. 2 is a block diagram of an implementation of a risk based insulin delivery rate converter 230. The risk based insulin delivery rate converter 230 comprises a comparator 235, a glycemic risk assessor 240, and an insulin delivery supervisor 245.

Inputs to the risk based insulin delivery rate converter 230 comprise continuous glucose monitoring (CGM) data 205, other sensed input data 210, insulin data 215, user input data 220, and configuration and/or setup input data 203. External process data 225 (e.g., a proposed basal rate and/or a proposed bolus rate) are also input to the insulin delivery supervisor 245 of the risk based insulin delivery rate converter 230. The output(s) 290 of the risk based insulin delivery rate converter 230 comprise approved basal rate and/or approved bolus rate.

The risk based insulin delivery rate converter 230 runs periodically and/or on demand to provide an approved basal rate and/or approved bolus rate for an upcoming time interval based on glycemic risk and model discrepancies. On/off criteria for the risk based insulin delivery rate converter 230 may be applied, e.g., when patient is initiating bolus or based on data credibility. In some embodiments, the risk based insulin delivery rate converter 230 runs periodically, e.g., every 5 minutes, whenever a new CGM value is received, etc.

The inputted CGM data 205 (e.g., glucose data), the other sensed input data 210, and the inputted insulin data 215 (e.g., previously dosed basal/bolus insulin with insulin on board (IOB) calculation) comprise the respective data up to the present time (i.e., up to now). In some embodiments, CGM data may be replaced with predicted data when CGM data is missing or not credible for a particular time interval. The user input data 220 may comprise data based on meals and/or exercise and/or other activity. Meals and exercise and other activity may be explicitly ignored or not allowed in some embodiments.

Additional inputs may include external process data 225, such as proposed basal rates and/or proposed bolus rates from an external process, which may include a pre-programmed basal profile (e.g., from an insulin pump), another AP algorithm (e.g., AID system), patient-initiated insulin delivery (basal or bolus), or the like. Systems and methods described herein convert proposed or externally derived bolus and/or basal rates into approved bolus and/or basal rates as described in more detail with regard to the insulin delivery supervisor 245. It should be noted that although arrows are shown at a high level into and out of a specific component, the inputs (and resulting outputs) to or from any process may be inputs into another process simultaneously, sequentially, after processing, or the like as may be appreciated by one skilled in the art.

It is contemplated that where an input includes a default basal insulin delivery profile, which typically defines a minimum amount of insulin per interval of time during continuous subcutaneous insulin infusion (CSII) over a 24 hour period, either defined by a patient or another system, this profile may have a feedback loop from the risk based insulin delivery rate converter 230 described herein. However, in some embodiments, the basal insulin delivery profile may be defined by the patient. While not wishing to be bound by theory, patients may modify basal rates in an attempt to compensate for missed boluses or missed meals, e.g., to minimize or avoid bolusing meals, which may negatively impact the techniques, processes, and/or algorithms provided herein. Accordingly, the systems and methods described herein are designed to supervise (i.e., convert, if necessary, and approve) proposed bolus and/or basal rates from external sources prior to outputting to the patient or system or other user, entity, component, module, or device.

The comparator 235 is configured to identify a discrepancy between differently derived estimations of metabolic and behavioral data (e.g., one with CGM data and one without CGM data) by quantifying the degree to which recent blood glucose measurements are inconsistent with recent insulin (and optionally additional data, e.g., carbohydrate records).

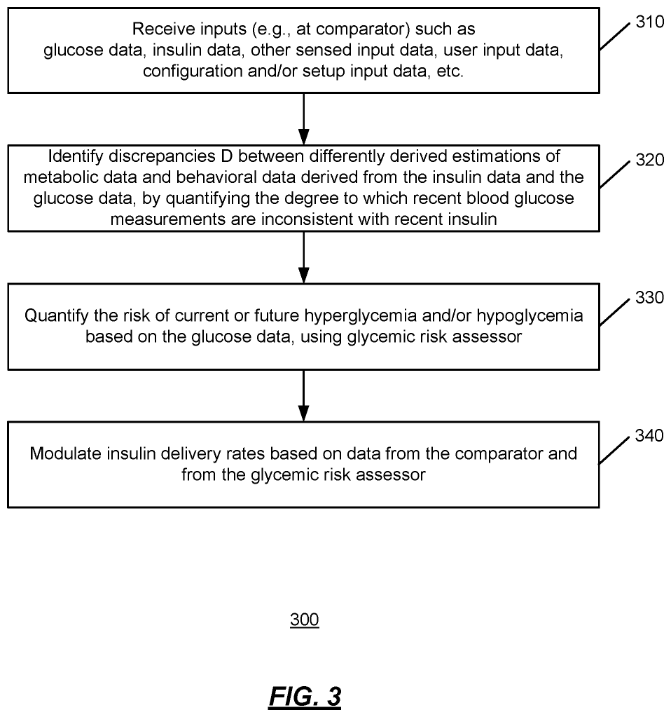

FIG. 3 is a flow diagram of an implementation of a method 300 of risk based insulin delivery rate conversion. The method 300 may be performed by the risk based insulin delivery rate converter 230.

At 310, inputs are received, e.g. at the comparator 235. The inputs may be comprise, for example, glucose data (e.g., CGM data 205), insulin data 215, other sensed input data 210, user input data 220, and/or configuration and/or setup input data 203, etc.

At 320, discrepancies (D) are identified between differently derived estimations of metabolic data and behavioral data derived from the insulin data and the glucose data, by quantifying the degree to which recent blood glucose measurements are inconsistent with recent insulin.

At 330, the risk of current or future hyperglycemia and/or hypoglycemia based on the glucose data is quantified, using the glycemic risk assessor 240.

At 340, insulin delivery rates are modified by the insulin delivery supervisor 245 based on data from the comparator 235 and from the glycemic risk assessor 240.

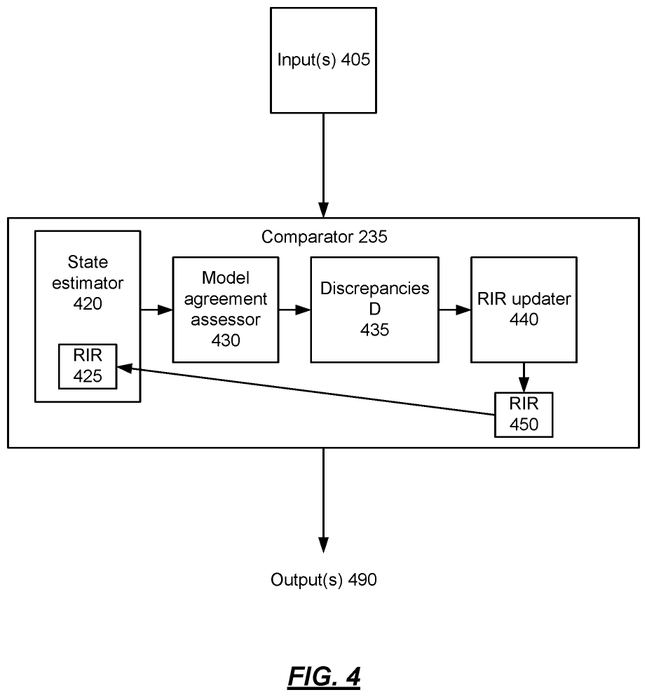

FIG. 4 is a block diagram of an implementation of a comparator, such as the comparator 235. As shown in FIG. 4, the comparator 235 comprises a state estimator 420, a model agreement assessor 430, and a reference insulin rate (RIR) updater 440.

The state estimator 420 may provide estimates of physiological and/or behavioral states of the patient based on CGM feedback, other sensed inputs, and/or user inputs. The state estimator 420 may include a model-based state observer such as a Kalman filter, or the like, which produces estimates of the physiological state (e.g., masses or concentrations of glucose, insulin, or other substances in various compartments) and/or behavioral state (e.g., eating or physical activity (now or in the recent past)) of the patient. The output from the state estimator 420 may be provided to the model agreement assessor 430, which then produces as one or more quantitative discrepancies D 435. The discrepancies may optionally be computed relative to a reference insulin rate (RIR) 425 in some embodiments. The output can be in the form of multiple vectors/matrices including the discrepancies D 435 or RIR 425, and the output may optionally comprise the history of the same in some embodiments.

In some embodiments, the state estimator 420 provides an estimate of one or more metabolic states, which may be based on an individualized physiological model to produce the following outputs: reconciled estimated metabolic inputs, estimated metabolic states of the patient for the duration of the input data, a numerical assessment of the credibility of the estimated states, and a numerical assessment of the credibility of the reconciled estimated metabolic inputs. The state estimator 420 is configured to receive the (optionally filtered) extrapolated inputs(s), any extracted condition and model parameters to the extent an individualized physiological model is being used by the estimator 420. A variety of estimators may be used depending on the implementation. In an implementation, the estimator 420 performs an open loop estimate of metabolic states into the future from the best estimates of the metabolic state vector at the beginning of the time series playing forward the individualized physiological model all the way to the end of the prediction horizon. Examples and implementations are described in U.S. application Ser. No. 17/096,785, entitled “JOINT STATE ESTIMATION PREDICTION THAT EVALUATES DIFFERENCES IN PREDICTED VS. CORRESPONDING RECEIVED DATA”, filed Nov. 12, 2020, inventor Stephen D. Patek, which is incorporated by reference herein in its entirety.

The model agreement assessor 430 may be or comprise a process or module that, for one or more state variables, evaluates discrepancies between two different models of metabolic states and/or behavioral states. In other words, the model agreement assessor 430 computes the discrepancies D 435 as a difference between a state estimator variable (based on all of the available data) and what the model would have predicted absent CGM data (open loop estimate) for the same variable, the discrepancy being the difference between the two versions of the variable. Notably, if metabolism could be perfectly modeled/predicted, then no CGM data would be needed. However, metabolism cannot be perfectly modeled/predicted, and the discrepancy can be used by the insulin delivery supervisor 245 described herein.

Exemplary state variables include: plasma glucose concentration or mass; interstitial glucose concentration or mass; glucose in other compartments of the body; rapid or long-acting insulin in subcutaneous tissue, in one or more compartments; insulin in blood plasma, the liver, or the periphery, resulting from subcutaneous or intravenous infusion or from endogenous secretion; states that describe the uptake, action, clearance of insulin or glucose in various compartments of the body; pharmacokinetic and/or pharmacodynamic states associated with medications; states associated with absorption of carbohydrates in meals; and the like.

In an implementation, a differential value is calculated as a measure of the degree to which recent CGM data are inconsistent with the physiological model used for state estimation. In one example, discrepancies between two different open loop predictions of metabolic and/or behavioral states are quantified into a delta, e.g., a comparison of state observer (including Kalman filter) estimates other states in other compartmental models to other open loop estimates. In one such implementation, for each internal state x, a delta Dx is computed (if possible) that would cause the open loop prediction to agree with CGM records. Examples are described in U.S. application Ser. No. 15/580,935, entitled “INSULIN MONITORING AND DELIVERY SYSTEM AND METHOD FOR CGM BASED FAULT DETECTION AND MITIGATION VIA METABOLIC STATE TRACKING”, filed Dec. 8, 2017, inventor Breton, which is incorporated by reference herein in its entirety. For example, a delta may be computed that is associated with the insulin action state of the model. However, the model agreement assessor 430 may quantify these discrepancies D 435 as a variance, a differential value, a delta variable, or the like. The continuity of CGM signals may be considered by the model agreement assessor 430 as well as values and/or trends of recent CGM data. In another embodiment, discrepancies between artificial intelligence (AI) and machine learning (ML) models using (i) all information including blood glucose (BG) and (ii) all information except BG are quantified. Other useful models include: a compartmental model of glucose-insulin dynamics that includes a state that corresponds to insulin action (e.g., the minimal model, or others), which may or may not be tuned to the patient's specific physiology; a Kalman filter to estimate the patient's insulin action state based on blood glucose measurements, recent insulin delivery and carb records; an open loop estimate of the insulin action state (using only recent insulin records); and the like.

The RIR updater 440 may determine an internal reference insulin rate 450. While it may overlap with a patient defined basal profile, the RIR 450 is distinct from a basal profile defined by a patient, doctor, or external process, which are specifically designed for compensation of meals and other behavioral events. Rather, the RIR 450 is an internal reference for what constitutes insulin that would achieve equilibrium. The RIR 450 may be a time averaged basal rate, adjusted over time, patient-dependent, fixed, zero, programmed, learned, prescribed, and/or the like. The RIR 450 may further be derived from total daily basal (total daily insulin or TDI), correction factor, and/or body mass index (BMI)/body weight. The RIR 450 may be updated every 5 minutes for example or defined by rate of data acquisition (from CGM). The RIR 450 may be used by the state estimator 425 to improve state estimations, BG prediction, and interpretation of discrepancies from the model agreement assessor 430. Accordingly, the RIR updater 440 may replace a time-varying basal rate as a reference for insulin delivery.

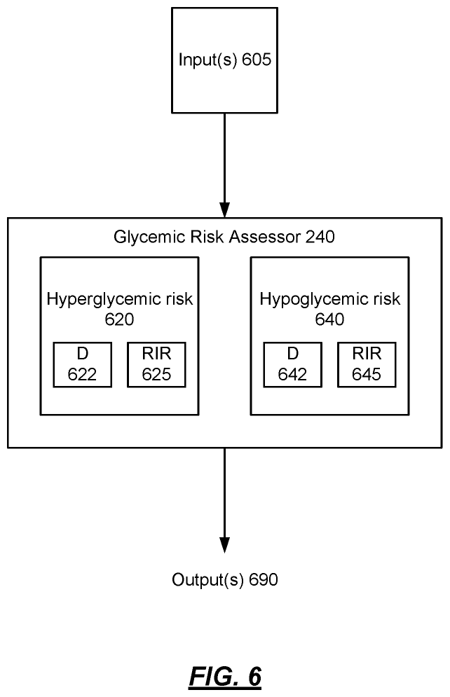

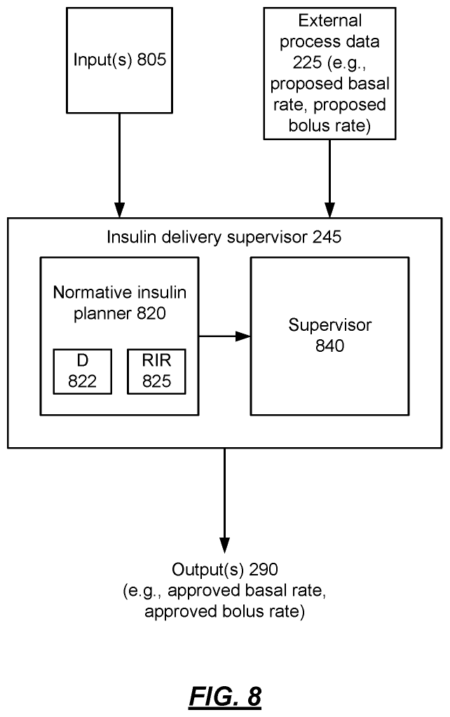

In some embodiments, the reference insulin rate RIR (450) is provided back to the state estimator 420 as a reference point (shown as RIR 425) for insulin in state estimation and prediction. Additionally or alternatively, in some embodiments, the discrepancies D (435) or the reference insulin rate RIR (450) are provided to the glycemic risk assessor 240 (shown, respectively, in FIG. 6 as D (in 622 and 642) and RIR (in 625 and 645), where RIR could serve as an alternative to the patient's preprogrammed basal rate profile as reference point for interpreting past insulin delivery in quantifying the risk of hypoglycemia or hyperglycemia. Additionally or alternatively, in some embodiments, the discrepancies D (435) or the reference insulin rate RIR (450) are provided to the insulin delivery supervisor 245 (shown, respectively, in FIG. 8 as D in 822 and RIR in 825), where RIR could serve as an alternative to the patient's preprogrammed basal rate profile as reference point in interpreting past and future proposed basal recommendations and/or proposed bolus recommendations from the external process data 225. The preprogrammed basal rate profile may have time-of-day features that would make the profile inappropriate as an insulin reference, e.g., partial control of regular meals via elevated basal rate.

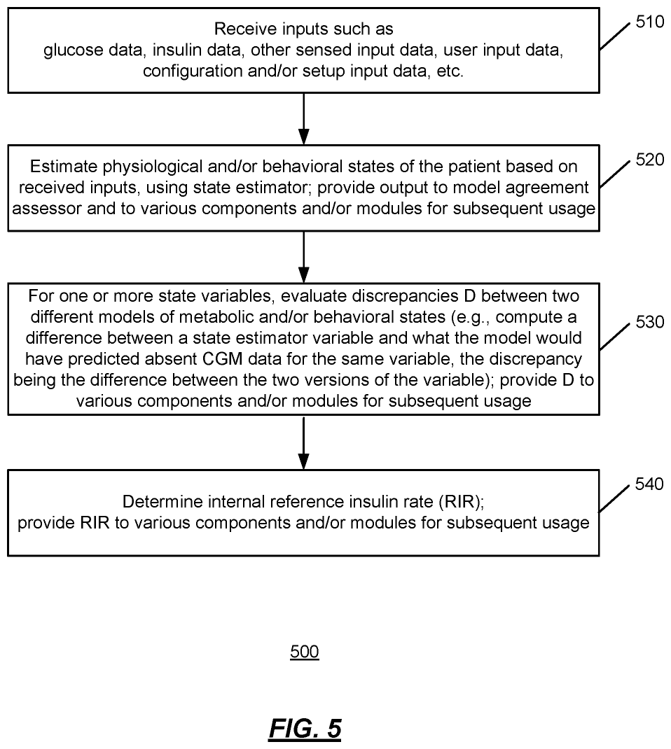

FIG. 5 is a flow diagram of an implementation of a method 500 of comparison for use with risk based insulin delivery rate conversion. The method 500 may be performed using the comparator 235.

At 510, inputs are received. The inputs may be comprise, for example, glucose data (e.g., CGM data 205), insulin data 215, other sensed input data 210, user input data 220, and/or configuration and/or setup input data 203, etc.

At 520, physiological and/or behavioral states of the patient are estimated based on received inputs, using a state estimator such as the state estimator 420. The output is provided to a model agreement assessor such as the model agreement assessor 430. Additionally or alternatively, the output may be provided to other components and/or modules for subsequent usage.

At 530, for one or more state variables, discrepancies D 435 between two different models of metabolic and/or behavioral states are evaluated. For example, a difference between a state estimator variable and what the model would have predicted absent CGM data for the same variable is computed, with the discrepancy being the difference between the two versions of the variable. The discrepancies D may be provided to other components and/or modules for subsequent usage.

At 540, an internal RIR is determined and provided to various components and/or modules (described further herein) for subsequent usage.

FIG. 6 is a block diagram of an implementation of a glycemic risk assessor, such as the glycemic risk assessor 240. The glycemic risk assessor 240 determines a hyperglycemic risk 620 and a hypoglycemic risk 640. The hyperglycemic risk 620 and the hypoglycemic risk 640 may be determined using the output of the state estimator 420. Additionally or alternatively, the hyperglycemic risk 620 and the hypoglycemic risk 640 may be determined using model agreement assessor discrepancies D 622 (for hyperglycemic risk) and D 642 (for hypoglycemic risk), respectively. Additionally or alternatively, the hyperglycemic risk 620 and the hypoglycemic risk 640 may be determined using the reference insulin rate RIR 625 and reference insulin rate RIR 645, respectively.

The glycemic risk assessor 240 quantifies the risk of current and future hyperglycemia and/or hypoglycemia, respectively. The glycemic risk assessor 240 calculates the level of risk from inputs such as blood glucose data, insulin data, user input data, state estimator outputs, RIR, and model agreement assessor discrepancies D, and may be based on predicted glucose in some embodiments. In some embodiments, glycemic risk (e.g., hypoglycemia and/or hyperglycemia calculation(s)) uses prediction/state estimation. The glycemic risk assessor 240 could be BG risk space quantification as in low blood glucose index (LBGI)/high blood glucose index (HBGI) and/or those examples and implementations described in U.S. Pat. No. 10,638,981, entitled “METHOD, SYSTEM AND COMPUTER READABLE MEDIUM FOR ASSESSING ACTIONABLE GLYCEMIC RISK”, inventor Stephen D. Patek, which is incorporated by reference herein in its entirety.

Each assessment for hyperglycemia and/or hypoglycemia respectively could be multivariate. This may include predicted BG (either for a specific horizon, or for a whole trajectory, or for a “hurricane track”). In an implementation, the glycemic risk assessor 240 could include delta described in Breton, such as those examples described in U.S. application Ser. No. 15/580,935, entitled “INSULIN MONITORING AND DELIVERY SYSTEM AND METHOD FOR CGM BASED FAULT DETECTION AND MITIGATION VIA METABOLIC STATE TRACKING”, filed Dec. 8, 2017, inventor Breton, which is incorporated by reference herein in its entirety. The glycemic risk assessor 240 may be configured to assess hyperglycemic risk alone or in combination with hypoglycemic risk, such as that described in U.S. application Ser. No. 14/659,500, entitled GLYCEMIC URGENCY ASSESSMENT AND ALERTS INTERFACE, filed Mar. 16, 2015, inventor Rack-Gomer, which is incorporated by reference herein in its entirety. Adjustment of the risk function may be parameterized. Normalized risk, such as that described in U.S. Pat. No. 10,638,981, entitled “METHOD, SYSTEM AND COMPUTER READABLE MEDIUM FOR ASSESSING ACTIONABLE GLYCEMIC RISK”, inventor Stephen D. Patek, which is incorporated by reference herein in its entirety, allows for parameterization of the shape of the risk function in a more natural way. Exemplary risk-based windows may be, e.g., over 5 minutes, over 30 minutes, a combination of basal/bolus, a time function, etc.

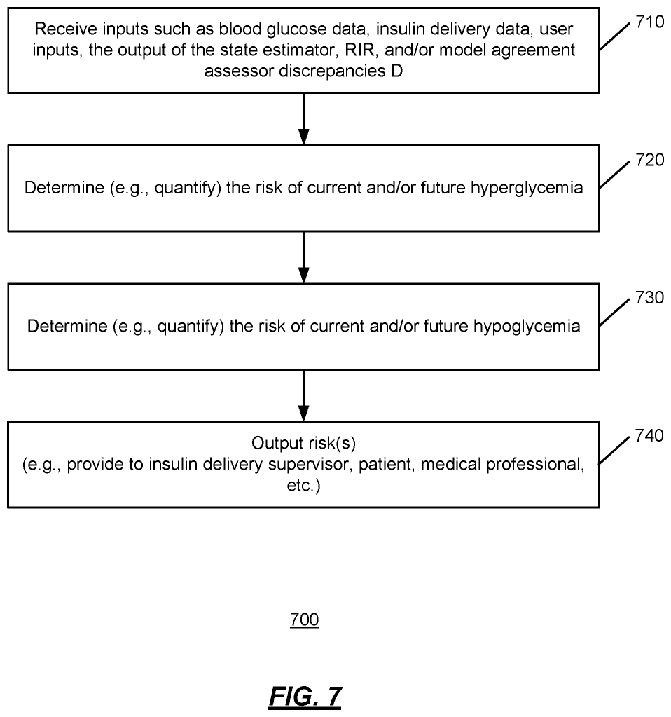

FIG. 7 is a flow diagram of an implementation of a method 700 of glycemic risk assessment for use with risk based insulin delivery rate conversion. The method 700 may be performed using the glycemic risk assessor 240.

At 710, inputs such as blood glucose data, insulin data, user input data, state estimator outputs, RIR, and/or model agreement assessor discrepancies D are received.

At 720, the risk of current and/or future hyperglycemia is determined (e.g., quantified).

At 730, the risk of current and/or future hypoglycemia is determined (e.g., quantified).

At 740, the risk(s) are outputted to an insulin delivery supervisor (e.g., the insulin delivery supervisor 245), a patient, a doctor or other medical professional or administrator, etc.

FIG. 8 is a block diagram of an implementation of an insulin delivery supervisor, such as the insulin delivery supervisor 245. The insulin delivery supervisor 245 comprises a normative insulin planner 820 and a supervisor 840.

The insulin delivery supervisor 245 modulates insulin delivery rates based on data from the comparator 235 and the glycemic risk assessor 240. The insulin delivery supervisor 245 further considers proposed bolus and/or basal rates from external processes, if available. Often, a proposed insulin rate (basal or bolus) is available, for example from conventional fully manual open loop therapy (CSII basal insulin profiles), decision support therapy (recommendation algorithms), control to range automated insulin delivery (AID), control to target AID, MPC, LQG, PID, or the like; however, the systems and methods described herein can function within a fully stand-alone algorithm as well some implementations.

Depending on the implementation, the insulin delivery supervisor 245 may include intensification of insulin (increased rate) based on hyperglycemic risk, attenuation of insulin based on hypoglycemic risk (decreased rate), or both. In some embodiments, the insulin delivery supervisor 245 calculates the insulin rate for a window of time over which the amount of needed insulin is to be delivered, wherein the window of time is determined from the level of glycemic risk, e.g., hyperglycemic risk or hypoglycemic risk. In some embodiments, the insulin rate is calculated based on the comparator 235, e.g., the model agreement assessor 430.

In an implementation, the insulin delivery supervisor 245 adjusts a proposed basal rate to an approved basal rate by adjusting the proposed value in response to the risk of hyperglycemia aiming to ensure that BG will remain below an upper envelope of acceptable values, wherein the upper envelope is a function of time (e.g., may be time of day or vary with respect to other parameters), as described further herein.

The normative insulin planner 820 considers the risk(s) of hyperglycemia and/or hypoglycemia (and optionally uses the estimated fault state and RIR 825) to determine a target trajectory of future insulin, which is converted into a proposed basal rate and/or bolus rate. The normative insulin planner 820 can function as an adjunctive layer for existing algorithm or within a stand-alone algorithm.

The normative insulin planner 820 determines an amount of insulin needed to minimize the discrepancy determined by the model agreement assessor 430 of the comparator 235. The amount could be a standard amount of insulin needed, such as ISOB (insulin that should be on board) as described in U.S. application Ser. No. 15/580,935, filed Dec. 8, 2017, published as US 2019/0254595 A1, entitled “INSULIN MONITORING AND DELIVERY SYSTEM AND METHOD FOR CGM BASED FAULT DETECTION AND MITIGATION VIA METABOLIC STATE TRACKING”, inventor Marc D. Breton, which is incorporated by reference herein in its entirety, or could be provided in terms of future plasma insulin or other physiological.

In one exemplary embodiment (e.g., described in U.S. application Ser. No. 15/580,935, incorporated by reference herein in its entirety), the states being assessed are IOB versus ISOB based on how much insulin it would take to get patient back to an upper BG envelope curve, wherein the upper BG envelope is a curve that depends upon the time of day, e.g., wherein curve value is high during day (e.g., 160 mg/dl) and at night it drops (e.g., to 120 mg/dl). In some embodiments, the upper BG envelope is calculated based on the current estimate of BG designed to allow BG to drop to an end-value over a window of time. In some embodiments, the systems and methods described herein impose a maximal curve value to ensure a substantial response to hyperglycemic risk to solve the problem of occasional tepid response to hyperglycemia. Notably, the upper envelope is used by ISOB as a target, but this is not the same as the target of the control algorithm for tuning insulin delivery. In some embodiments, the normative insulin planner 820 computes ISOB based on a target defined by an upper BG envelope curve that is generated on demand based on estimated BG. In some embodiments, the envelope is determined from a sleep profile; however, this is not a requirement and may in fact be avoided in certain implementations. In some embodiments, ISOB may be computed as a function of both upper and lower BG envelopes, e.g., ISOB could be computed to achieve a BG somewhere in between the hyperglycemia upper envelope curve value and a low-BG envelope consistent with an insulin shut off threshold or logic.

In some embodiments, insulin may be expressed directly in terms of subcutaneous insulin delivery (e.g., see ISOB as described in U.S. application Ser. No. 15/580,935, incorporated by reference herein in its entirety) relative to user-provided basal rate profile or relative to reference insulin rate (RIR). In some embodiments, the output of the normative insulin planner can be based on a BG upper envelope curve or other mechanisms for optimizing the nominal insulin trajectory of the patient.

The time window (sometimes referred to as a “rate window”) used for determining insulin delivery rate (to account for level of agreement or disagreement) may be a function of risk of glycemia (e.g., hyperglycemia or hypoglycemia) and is therefore variable. For example, when there is a high hyperglycemic risk, then the full amount of insulin needed may be delivery at the fastest rate possible, i.e., as a bolus.

The supervisor 840 may be combined with or separated from the normative insulin planner 820. The supervisor 840 reconciles proposed basal rate (and optionally proposed bolus rate) from external sources (e.g., the external process data 225) with insulin needs identified by the normative insulin planner 820 to determine an approved basal rate (and/or bolus) for the next periodic update. U.S. application Ser. No. 15/580,935, incorporated by reference herein in its entirety, describes an implementation of determining insulin needs by calculating ISOB and comparing to IOB; however, other methods of determining an insulin need may be used.

The supervisor 840, in addition to inputs from the original inputs, may process the output of the state estimator 420, the output of the model agreement assessor 430 (i.e., the discrepancies D), the output of the RIR updater 440 (i.e., RIR 450), and may further include inputs from externally derived processes that describe basal insulin and optionally bolus insulin (i.e., the external process data 225). Accordingly, the supervisor 840 may be useful to reconcile external processes with the systems and methods described herein for insulin planning.

In some embodiments, the insulin delivery supervisor 245 may convert a bolus recommendation into a combination of bolus and basal, for example, an amount delivered at a maximum rate and an amount be delivered as an elevated basal rate over some period of time. The conversion may be based on the state of the system and glycemic risk and may feedback into a previous step and/or module of the methods and/or systems described herein. In some implementations, the conversion may be informed by when the next decision may be made. In some implementations, the risk based insulin delivery rate converter 230 takes output of any open loop or closed loop artificial pancreas algorithm designed to produce a rate of insulin delivery and converts the rate into mixture of basal rate and discrete boluses. In some implementations, the discrete (correction) boluses are coordinated with basal rates based on hyperglycemic risk, i.e., the insulin delivery supervisor 245 converts the recommended correction boluses into rates, wherein the rate window is computed as a function of hyperglycemic risk (e.g., rather than a fixed rate window of 30 minutes or the like).

In one example, predicted blood glucose is used to calculate hyperglycemic risk, which is used by the model agreement assessor 430 to quantify a variance between blood glucose and/or insulin states, wherein the greater the risk of hyperglycemia results in a shorter rate window; in other words, at the highest level of hyperglycemia, the insulin amount required is delivered as a discrete bolus. Accordingly, the rate window is variable such that as a higher risk of hyperglycemic is computed, the rate window will come nearer 5 minutes (or whatever the period rate of refresh of the data acquisition and/or controller update). As one example, when risk based insulin delivery rate converter 230 computed a difference between ISOB and IOB to be 3 units, it can be delivered in 5 minutes at high levels of hyperglycemic risk, but over 30 minutes at low levels of hyperglycemic risk. Notably here, pre-intervention hyperglycemic risk is used to convert ISOB into a rate of insulin delivery that will apply until the next controller update, such that when there is high hyperglycemic risk, ISOB is delivered as a discrete bolus. However, conversion of ISOB into a rate could be informed by both pre- and post-intervention predicted BG in some embodiments. In contrast to standard model predictive control (MPC), the rate value is a modification of the ISOB value, which is not the result of an optimization.

Although the example above described the use of hyperglycemic risk, the conversion of ISOB into a rate could be informed by both hyperglycemic risk and hypoglycemic risk.

As shown in the formula below, the rate window may be the denominator of a discrete bolus conversion into a rate based on hyperglycemic risk:

insulin delivery rate=(amount of insulin needed based on the level of agreement)/(risk based window of time over which the amount of needed insulin is to be delivered).

The resulting recommendation from the supervisor 840 can thus be large enough to achieve the effect of a discrete correction and/or meal bolus, or small enough to include low basal rates of delivery.

In some embodiments, the aggressiveness of the insulin delivery supervisor 245 may be constrained based on an assessment of the patient's daily total insulin requirement (TDI). For example, the parameters needed to compute appropriate responses to differences between IOB and ISOB could be constrained as a function of TDI. An ongoing revision of TDI modulates how aggressive the normative insulin planner is allowed to be. A saturated value of correction factor may be used as a separate check on how aggressive the control algorithm is allowed to be. Limits on correction factor may be implemented here.

The output(s) 290 from the insulin delivery supervisor 245 include an approved basal rate and optionally an approved bolus rate. The output(s) 290 may also comprise a message sent to a patient, a doctor or other medical professional or administrator, display, computing device, etc. For example, a predicted BG trajectory may be displayed with a description of the uncertainty. A recommended value or amount of insulin delivery may be provided or described for a specific time interval and/or with respect to various conditions (e.g., “if”, “when”, “based on”, “time in range outcomes without meal announcement”, etc.).

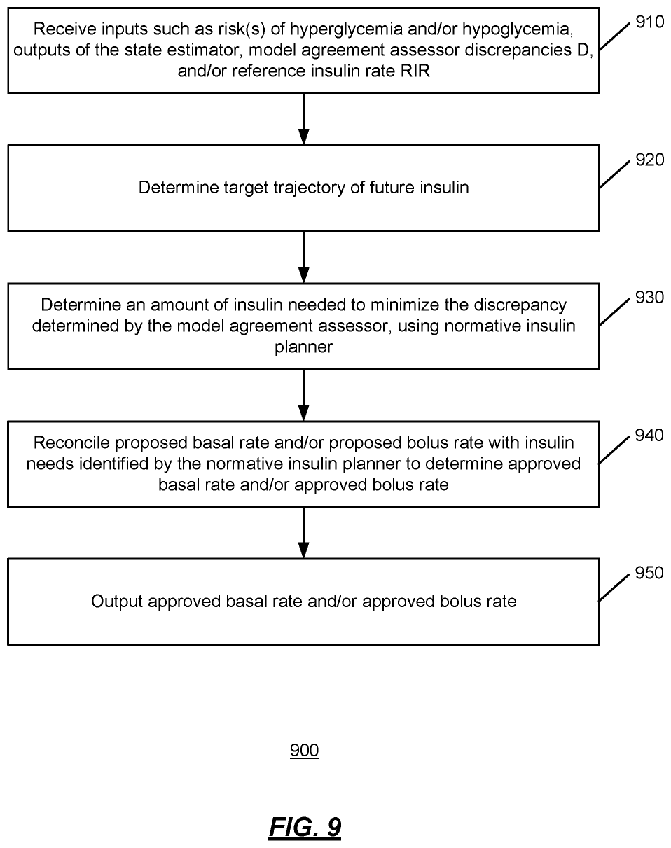

FIG. 9 is a flow diagram of an implementation of a method 900 of insulin delivery supervision for use with risk based insulin delivery rate conversion. The method 900 may be performed using the insulin delivery supervisor 245.

At 910, inputs such as risk(s) of current and/or future hyperglycemia and/or hypoglycemia, outputs of the state estimator 420, the outputs of the model agreement assessor 430 (i.e., the discrepancies D), the output of the RIR updater 440 (i.e. RIR 450), and inputs from externally derived processes that describe basal insulin and optionally bolus insulin are received.

At 920, a target trajectory of future insulin is determined.

At 930, an amount of insulin is determined that is needed to minimize the discrepancies D from the model agreement assessor 430 and/or minimize hyperglycemic risk, using a normative insulin planner.

At 940, the proposed basal rate and/or proposed bolus rate is reconciled with insulin needs identified by the normative insulin planner to determine approved basal rate and/or approved bolus rate.

At 950, an approved basal rate and/or an approved bolus rate is outputted, e.g., to a delivery device, a patient, a doctor or other medical professional or administrator, a display device, a computing device, etc.

### Example 1—Supervising Conventional Insulin Pump Therapy Implementation

In an implementation, systems and methods described herein are operatively used with an insulin pump therapy system (external process) with user-programmed basal rate profile and functional pre-meal insulin boluses computed using an estimate of carbs, a carbohydrate ratio, a correction factor, and JOB. In this example, the system/method runs as follows.

The comparator 235 quantitatively reconciles open loop and CGM based estimates of the current metabolic state vector in different ways, including one or more of: by attributing level of agreement (or disagreement) to failure to deliver insulin (e.g. pump occlusion)→setting of a pump fault state estimate; by attributing agreement/disagreement to unexpected low/high “insulin action”→recognition of the fact that the insulin sensitivity parameter is too low/high→incremental adjustment of model agreement assessor 430 discrepancy for insulin action (D); and/or by attributing quantified agreement/disagreement to an unannounced meal (or a meal with greater carb content than acknowledged by the patient).

The glycemic risk assessor 240 estimates a quantitative value of hyperglycemic risk and/or hypoglycemic risk applicable over a specified planning horizon. It is noted that this assumes no further interventions from the user.

The insulin delivery supervisor 245 (knowing the user-programmed basal rate profile from the operably connected insulin delivery device) estimates the effect of the basal rate profile over the specified planning horizon, optionally seeing a current bolus request from the patient, and without future intervention assumptions from the patient, may determine to: modify the current bolus request (if there is one), or issue an unrequested insulin bolus; modify the basal rate profile for the duration of the planning horizon; and/or specify that a bolus be delivered at some future point in the planning horizon.

In one exemplary circumstance (set of conditions) with this implementation, when the preprogrammed basal profile is elevated with respect to the patient's fasting basal profile (or RIR), that elevated basal rate may represent an attempt by the user to treat an unannounced meal in part with basal insulin delivery. In this case, the supervisor serves to accelerate the effect of that elevated basal rate by converting part of it to a discrete bolus.

In another exemplary circumstance (set of conditions) with this implementation, when: the comparator 235 may determine that either (i) unannounced/underestimated carbs exist or (ii) reduced insulin sensitivity are the most likely explanation of model disagreement; the glycemic risk assessor 240 may then estimate an elevated, clinically significant, risk R of hyperglycemia; and the user recently specified a discrete bolus B, then: the insulin delivery supervisor 245 either: delivers a bolus now equal to B plus a fraction F of the total amount of insulin associated with the preprogrammed basal rate profile for the specified planning horizon T, where the fraction is computed as a function of the estimated risk R of hyperglycemia, (for example F=k*R/(1+k*R), where k is a parameter) and determines to deliver the remaining fraction (1−F) of insulin as a new reduced temporary basal rate for the duration of the specified planning horizon T; or may defer to the user's judgement and only deliver B and wait for a future opportunity for the supervisor to preemptively convert basal insulin into a bolus, which may be dependent on a variety of factors, such as credibility of data.

In yet another exemplary circumstance (set of conditions) with this implementation, when: the comparator 235 estimates that unexpectedly high insulin action (suggesting momentary high insulin sensitivity) is the most likely explanation of model disagreement; the glycemic risk assessor 240 estimates an elevated, clinically significant, risk R of hypoglycemia; and the user has recently input a discrete bolus B≥0, then the insulin delivery supervisor 245 may set a temporary basal rate over a specified planning horizon based on the risk. For example, based on a desire to achieve a particular IOB, knowing the user bolus B the basal rate could be set to achieve that IOB within a specified timeframe, where both the target IOB and the timeframe are computed as a function of the estimated risk of hypoglycemia. Additionally or alternatively, the insulin delivery supervisor 245 may alert the user about the bolus, suggesting that without additional carbohydrates the bolus may exacerbate the risk of hypoglycemia.

### Example 2—Supervising an Automated Insulin Delivery (AID) Algorithm

In this second exemplary implementation, the systems and methods described herein are operably used with an automated insulin delivery therapy system (external process) including automated adjustment of basal rates and/or automated insulin boluses, with or without the opportunity for patients to request boluses. The comparator 235 quantitatively reconciles open loop and CGM based estimates of the current metabolic state vector as described in Example 1 and further based on the estimated RIR.

The glycemic risk assessor 240 estimates a quantitative value of and/or hypoglycemic risk applicable over a specified planning horizon (note: assumes no further interventions from the user).

The insulin delivery supervisor 245, based on the patient's RIR, responsive to a recommendation of higher-than-RIR basal rate and/or an insulin bolus request/recommendation, and assuming no future intervention from the patient, may determine to: modify the current bolus request/recommendation (if there is one), or introduce a new bolus; modify the AID basal rate recommendation; and/or specify that a bolus be delivered at some future point in the specified planning horizon.

In one exemplary circumstance (set of conditions) with this second implementation, when: the comparator 235 determines either (i) unannounced/underestimated carbs or (ii) reduced insulin sensitivity is the most likely explanation of model disagreement; the glycemic risk profiler estimates an elevated, clinically significant, risk R of hyperglycemia; and no bolus recommendation or request is present, then the insulin delivery supervisor 245 may deliver a bolus now equal to a fraction F of the total amount of insulin associated with the AID-recommended basal rate profile for the specified planning horizon T, where the fraction is computed as a function of the estimated risk R of hyperglycemia (e.g., F=k*R/(1+k*R), where k is a parameter) and deliver the remaining fraction (1−F) of insulin associated with the AID basal rate recommendation as a new reduced basal rate. Additionally or alternatively, the bolus introduced above could be computed as a function of the difference between the AID-recommended basal rate and the patient's RIR.

In another exemplary circumstance (set of conditions) with this second implementation, when: the comparator 235 recognizes that either (i) unannounced/underestimated carbs or (ii) reduced insulin sensitivity are the most likely explanation of model disagreement; the glycemic risk assessor 240 estimates an elevated, clinically significant, risk R of hyperglycemia; and the user just specified a discrete bolus B, then the insulin delivery supervisor 245 determines to deliver a bolus now equal to B plus a fraction F of the total amount of insulin associated with the AID-recommended basal rate profile for the specified planning horizon T, where the fraction is computed as a function of the estimated risk R of hyperglycemia, e.g., F=k*R/(1+k*R), where k is a parameter; and to deliver the remaining fraction (1−F) of insulin associated with the AID basal rate recommendation as a new reduced basal rate. Alternatively, the insulin delivery supervisor 245 may defer to the user's judgement and only deliver B and wait for a future opportunity for the supervisor to preemptively convert basal insulin into a bolus, for example, which may be based on data credibility or a fail-safe feature.

In yet another exemplary circumstance (set of conditions) with this second implementation, when: the comparator 235 recognizes that unexpectedly high insulin action (suggesting momentary high insulin sensitivity) is the most likely explanation of model disagreement; the glycemic risk assessor 240 estimates an elevated, clinically significant, risk R of hypoglycemia; and the user just specified a discrete bolus B≥0, then the insulin delivery supervisor 245 determines to set a temporary basal rate over a specified planning horizon, e.g., based on a desire to achieve a particular JOB, knowing the user bolus B the basal rate could be set to achieve that IOB within a specified timeframe, where both the target IOB and the timeframe are computed as a function of the estimated risk of hypoglycemia. If it turns out that the reduced basal rate was necessary (e.g., to compensate for unannounced carbs), then the difference can be introduced later as either as a compensated elevated basal rate or as discrete bolus. Additionally or alternatively, the supervisor may determine to leave the bolus B unchanged but, unless B=0, display/alert the user about the bolus, suggesting that without additional carbohydrates the bolus may exacerbate the risk of hypoglycemia.

FIG. 10 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 10, an exemplary system for implementing aspects described herein includes a computing device, such as computing device 1000. In its most basic configuration, computing device 1000 typically includes at least one processing unit 1002 and memory 1004. Depending on the exact configuration and type of computing device, memory 1004 may be volatile (such as random access memory (RAM)), non-volatile (such as read-only memory (ROM), flash memory, etc.), or some combination of the two. This most basic configuration is illustrated in FIG. 10 by dashed line 1006.

Computing device 1000 may have additional features/functionality. For example, computing device 1000 may include additional storage (removable and/or non-removable) including, but not limited to, magnetic or optical disks or tape. Such additional storage is illustrated in FIG. 10 by removable storage 1008 and non-removable storage 1010.

Computing device 1000 typically includes a variety of computer readable media. Computer readable media can be any available media that can be accessed by the device 1000 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 1004, removable storage 1008, and non-removable storage 1010 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1000. Any such computer storage media may be part of computing device 1000.

Computing device 1000 may contain communication connection(s) 1012 that allow the device to communicate with other devices. Computing device 1000 may also have input device(s) 1014 such as a keyboard, mouse, pen, voice input device, touch input device, etc. Output device(s) 1016 such as a display, speakers, printer, etc. may also be included. All these devices are well known in the art and need not be discussed at length here.

In an implementation, a risk based insulin delivery rate converter comprises: a comparator that is configured to receive insulin data and glucose data, and comprises a model agreement assessor configured to identify a discrepancy between differently derived estimations of metabolic data and behavioral data derived from the insulin data and the glucose data, by quantifying the degree to which recent blood glucose measurements are inconsistent with recent insulin; a glycemic risk assessor configured to quantify the risk of at least one of current or future hyperglycemia or hypoglycemia based on the glucose data; and an insulin delivery supervisor configured to modulate insulin delivery rates based on data from the comparator and the glycemic risk assessor.

In an implementation, a risk based insulin delivery rate conversion method comprises: receiving insulin data and glucose data at a comparator; identifying a discrepancy between differently derived estimations of metabolic data and behavioral data derived from the insulin data and the glucose data, using a model agreement assessor of the comparator, by quantifying the degree to which recent blood glucose measurements are inconsistent with recent insulin; quantifying the risk of at least one of current or future hyperglycemia or hypoglycemia based on the glucose data, using a glycemic risk assessor; and modulating insulin delivery rates based on data from the comparator and the glycemic risk assessor, using an insulin delivery supervisor.

In an implementation, a system comprises: at least one processor; and a non-transitory computer readable medium comprising instructions that, when executed by the at least one processor, cause the system to: receive insulin data and glucose data at a comparator; identify a discrepancy between differently derived estimations of metabolic data and behavioral data derived from the insulin data and the glucose data, using a model agreement assessor of the comparator, by quantifying the degree to which recent blood glucose measurements are inconsistent with recent insulin; quantify the risk of at least one of current or future hyperglycemia or hypoglycemia based on the glucose data, using a glycemic risk assessor; and modulate insulin delivery rates based on data from the comparator and the glycemic risk assessor, using an insulin delivery supervisor.

Implementations may include some or all of the following features. The computer readable medium further comprises instructions that, when executed by the at least one processor, cause the system to evaluate discrepancies between two different models of metabolic states or behavioral states, using the model agreement assessor, and provide the discrepancies as output for subsequent usage. The computer readable medium further comprises instructions that, when executed by the at least one processor, cause the system to quantify discrepancies between two different open loop predictions of metabolic states or behavioral states as a variance, using the model agreement assessor. The computer readable medium further comprises instructions that, when executed by the at least one processor, cause the system to estimate at least one of physiological states or behavioral states of the patient based on at least one of continuous glucose monitoring (CGM) feedback, other sensed inputs, or user inputs, using a state estimator of the comparator, and provide an output to the model agreement assessor. The state estimates are used by the model agreement assessor. The computer readable medium further comprises instructions that, when executed by the at least one processor, cause the system to assess hyperglycemic risk by the glycemic risk assessor, which is used to modulate a time window over which the insulin rate is calculated by the insulin delivery supervisor. The insulin delivery supervisor considers at least one of proposed bolus rates or basal rates from external processes. The insulin delivery supervisor calculates the insulin rate for a window of time over which the amount of needed insulin is to be delivered. The window of time is determined from the level of glycemic risk quantified by the glycemic risk assessor. The computer readable medium further comprises instructions that, when executed by the at least one processor, cause the system to determine an amount of insulin needed to minimize the discrepancy determined by the comparator, by a insulin planner of the insulin delivery supervisor. The computer readable medium further comprises instructions that, when executed by the at least one processor, cause the system to reconcile proposed basal rate from external sources with insulin needs identified by the insulin planner to determine an approved basal rate for the next periodic update, by a supervisor of the insulin delivery supervisor. The computer readable medium further comprises instructions that, when executed by the at least one processor, cause the system to convert the approved basal rate into a mixture of basal rate and discrete boluses, using the insulin delivery supervisor.

In an implementation, a risk based insulin delivery rate converter comprises: a comparator that is configured to receive insulin data and glucose data, and comprises a model agreement assessor configured to identify a discrepancy between differently derived estimations of metabolic data and behavioral data derived from the insulin data and the glucose data, by quantifying the degree to which recent blood glucose measurements are inconsistent with recent insulin; a glycemic risk assessor configured to quantify the risk of at least one of current or future hyperglycemia or hypoglycemia based on the glucose data; an insulin delivery supervisor configured to modulate insulin delivery rates based on data from the comparator and the glycemic risk assessor; and a reference insulin rate (RIR) updater configured to determine a RIR, wherein the RIR is an internal reference for insulin that would achieve equilibrium.

In an implementation, a risk based insulin delivery rate conversion method comprises: receiving insulin data and glucose data at a comparator; identifying a discrepancy between differently derived estimations of metabolic data and behavioral data derived from the insulin data and the glucose data, using a model agreement assessor of the comparator, by quantifying the degree to which recent blood glucose measurements are inconsistent with recent insulin; quantifying the risk of at least one of current or future hyperglycemia or hypoglycemia based on the glucose data, using a glycemic risk assessor; modulating insulin delivery rates based on data from the comparator and the glycemic risk assessor, using an insulin delivery supervisor; and determining a reference insulin rate (RIR) using a RIR updater, wherein the RIR is an internal reference for insulin that would achieve equilibrium.

In an implementation, a system comprises: at least one processor; and a non-transitory computer readable medium comprising instructions that, when executed by the at least one processor, cause the system to: receive insulin data and glucose data at a comparator; identify a discrepancy between differently derived estimations of metabolic data and behavioral data derived from the insulin data and the glucose data, using a model agreement assessor of the comparator, by quantifying the degree to which recent blood glucose measurements are inconsistent with recent insulin; quantify the risk of at least one of current or future hyperglycemia or hypoglycemia based on the glucose data, using a glycemic risk assessor; modulate insulin delivery rates based on data from the comparator and the glycemic risk assessor, using an insulin delivery supervisor; and determine a reference insulin rate (RIR) using a RIR updater, wherein the RIR is an internal reference for insulin that would achieve equilibrium.

Implementations may include some or all of the following features. The RIR updater is comprised within the comparator. The RIR is used by the comparator. The glycemic risk assessor is configured to receive the RIR and use the RIR to quantify the risk of at least one of current or future hyperglycemia or future hypoglycemia. The insulin delivery supervisor is configured to receive the RIR and use the RIR to determine a target trajectory of future insulin and an amount of insulin needed to minimize the discrepancy. The insulin delivery supervisor is further configured to receive discrepancy data and use discrepancy data to determine the target trajectory of future insulin and the amount of insulin needed to minimize the discrepancy. The computer readable medium further comprises instructions that, when executed by the at least one processor, cause the system to evaluate discrepancies between two different models of metabolic states or behavioral states, using the model agreement assessor, and provide the discrepancies as output for subsequent usage. The computer readable medium further comprises instructions that, when executed by the at least one processor, cause the system to quantify discrepancies between two different open loop predictions of metabolic states or behavioral states as a variance, using the model agreement assessor. The computer readable medium further comprises instructions that, when executed by the at least one processor, cause the system to estimate at least one of physiological states or behavioral states of the patient based on at least one of continuous glucose monitoring (CGM) feedback, other sensed inputs, or user inputs, using a state estimator of the comparator, and providing an output to the model agreement assessor. The state estimates are used by the model agreement assessor. The computer readable medium further comprises instructions that, when executed by the at least one processor, cause the system to assess hyperglycemic risk by the glycemic risk assessor, which is used to modulate a time window over which the insulin rate is calculated by the insulin delivery supervisor. The insulin delivery supervisor considers at least one of proposed bolus rates or basal rates from external processes. The insulin delivery supervisor calculates the insulin rate for a window of time over which the amount of needed insulin is to be delivered. The window of time is determined from the level of glycemic risk quantified by the glycemic risk assessor. The computer readable medium further comprises instructions that, when executed by the at least one processor, cause the system to determine an amount of insulin needed to minimize the discrepancy determined by the comparator, by a insulin planner of the insulin delivery supervisor. The computer readable medium further comprises instructions that, when executed by the at least one processor, cause the system to reconcile proposed basal rate from external sources with insulin needs identified by the insulin planner to determine an approved basal rate for the next periodic update, by a supervisor of the insulin delivery supervisor. The computer readable medium further comprises instructions that, when executed by the at least one processor, cause the system to convert the approved basal rate into a mixture of basal rate and discrete boluses, using the insulin delivery supervisor.

In an implementation, a method comprises: receiving a plurality of inputs at a comparator; identifying discrepancies between differently derived estimations of metabolic data and behavioral data derived from inputs; quantifying the risk of current or future hyperglycemia or hypoglycemia based on the glucose data, using a glycemic risk assessor; and modulating insulin delivery rates, using an insulin delivery supervisor, based on data from the comparator and from the glycemic risk assessor.

In an implementation, a system comprises: a comparator configured to receive a plurality of inputs and identify discrepancies between differently derived estimations of metabolic data and behavioral data derived from the inputs; a glycemic risk assessor configured to quantify the risk of current or future hyperglycemia or hypoglycemia based on the glucose data; and an insulin delivery supervisor configured to modulate insulin delivery rates based on data from the comparator and from the glycemic risk assessor.

In an implementation, a system comprises: at least one processor; and a non-transitory computer readable medium comprising instructions that, when executed by the at least one processor, cause the system to: receive a plurality of inputs at a comparator; identify discrepancies between differently derived estimations of metabolic data and behavioral data derived from inputs; quantify the risk of current or future hyperglycemia or hypoglycemia based on the glucose data, using a glycemic risk assessor; and modulate insulin delivery rates, using an insulin delivery supervisor, based on data from the comparator and from the glycemic risk assessor.

Implementations may include some or all of the following features. The inputs comprise at least one of glucose data, insulin data, sensed input data, or user input data. Identifying the discrepancies comprises quantifying the degree to which recent blood glucose measurements are inconsistent with recent insulin. The computer readable medium further comprises instructions that, when executed by the at least one processor, cause the system to, at the comparator: estimate physiological or behavioral states of the patient based on received inputs, using a state estimator; provide an output to a model agreement assessor; for one or more state variables, evaluate discrepancies between two different models of metabolic or behavioral states; and output the discrepancies. Evaluating the discrepancies comprises computing a difference between a state estimator variable and what the model would have predicted absent continuous glucose monitoring (CGM) data for the same variable, the discrepancy being the difference between the two versions of the variable. The computer readable medium further comprises instructions that, when executed by the at least one processor, cause the system to determine an internal reference insulin rate (RIR) and output the RIR. The computer readable medium further comprises instructions that, when executed by the at least one processor, cause the system to, at the glycemic risk assessor: determine a risk of at least one of current or future hyperglycemia; determine a risk of at least one of current or future hypoglycemia; and output the risk of at least one of current or future hyperglycemia and the risk of at least one of current or future hypoglycemia. The computer readable medium further comprises instructions that, when executed by the at least one processor, cause the system to, at the insulin delivery supervisor: determine a target trajectory of future insulin; determine an amount of insulin needed to minimize a discrepancy determined by a model agreement assessor, using a normative insulin planner; reconcile a proposed basal rate or a proposed bolus rate with insulin needs identified by the normative insulin planner to determine an approved basal rate or an approved bolus rate; and output the approved basal rate or the approved bolus rate.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method comprising:
receiving a plurality of inputs at a comparator;
identifying discrepancies between differently derived estimations of metabolic data and behavioral data derived from inputs;
quantifying the risk of current or future hyperglycemia or hypoglycemia based on the glucose data, using a glycemic risk assessor; and
modulating insulin delivery rates, using an insulin delivery supervisor, based on data from the comparator and from the glycemic risk assessor.

2. The method of claim 1, wherein the inputs comprise at least one of glucose data, insulin data, sensed input data, or user input data.

3. The method of claim 2, wherein identifying the discrepancies comprises quantifying the degree to which recent blood glucose measurements are inconsistent with recent insulin.

4. The method of claim 2, further comprising, at the comparator:
estimating physiological or behavioral states of the patient based on received inputs, using a state estimator;
providing an output to a model agreement assessor;
for one or more state variables, evaluating discrepancies between two different models of metabolic or behavioral states; and
outputting the discrepancies.

5. The method of claim 4, wherein evaluating the discrepancies comprises computing a difference between a state estimator variable and what the model would have predicted absent continuous glucose monitoring (CGM) data for the same variable, the discrepancy being the difference between the two versions of the variable.

6. The method of claim 4, further comprising determining an internal reference insulin rate (RIR) and outputting the RIR.

7. The method of claim 6, further comprising at the glycemic risk assessor:
determining a risk of at least one of current or future hyperglycemia;
determining a risk of at least one of current or future hypoglycemia; and
outputting the risk of at least one of current or future hyperglycemia and the risk of at least one of current or future hypoglycemia.

8. The method of claim 7, further comprising at the insulin delivery supervisor:
determining a target trajectory of future insulin;
determining an amount of insulin needed to minimize a discrepancy determined by a model agreement assessor, using a normative insulin planner;
reconciling a proposed basal rate or a proposed bolus rate with insulin needs identified by the normative insulin planner to determine an approved basal rate or an approved bolus rate; and
outputting the approved basal rate or the approved bolus rate.

9. A system comprising:
a comparator configured to receive a plurality of inputs and identify discrepancies between differently derived estimations of metabolic data and behavioral data derived from the inputs;
a glycemic risk assessor configured to quantify the risk of current or future hyperglycemia or hypoglycemia based on the glucose data; and
an insulin delivery supervisor configured to modulate insulin delivery rates based on data from the comparator and from the glycemic risk assessor.

10. The system of claim 9, wherein the inputs comprise at least one of glucose data, insulin data, sensed input data, or user input data.

11. The system of claim 10, wherein identifying the discrepancies comprises quantifying the degree to which recent blood glucose measurements are inconsistent with recent insulin.

12. The system of claim 9, further comprising the comparator configured to:
estimate physiological or behavioral states of the patient based on received inputs, using a state estimator;
provide an output to a model agreement assessor;
for one or more state variables, evaluate discrepancies between two different models of metabolic or behavioral states; and
output the discrepancies.

13. The system of claim 12, wherein evaluating the discrepancies comprises computing a difference between a state estimator variable and what the model would have predicted absent continuous glucose monitoring (CGM) data for the same variable, the discrepancy being the difference between the two versions of the variable.

14. The system of claim 12, further comprising an reference insulin rate (RIR) updater configure to determining an internal RIR and output the RIR.

15. The system of claim 14, further comprising the glycemic risk assessor configured to:
determine a risk of at least one of current or future hyperglycemia;
determine a risk of at least one of current or future hypoglycemia; and
output the risk of at least one of current or future hyperglycemia and the risk of at least one of current or future hypoglycemia.

16. The system of claim 15, further comprising the insulin delivery supervisor configured to:
determine a target trajectory of future insulin;
determine an amount of insulin needed to minimize a discrepancy determined by a model agreement assessor, using a normative insulin planner;
reconcile a proposed basal rate or a proposed bolus rate with insulin needs identified by the normative insulin planner to determine an approved basal rate or an approved bolus rate; and
output the approved basal rate or the approved bolus rate.

17. A system comprising:
at least one processor; and
a non-transitory computer readable medium comprising instructions that, when executed by the at least one processor, cause the system to:
receive a plurality of inputs at a comparator;
identify discrepancies between differently derived estimations of metabolic data and behavioral data derived from inputs;
quantify the risk of current or future hyperglycemia or hypoglycemia based on the glucose data, using a glycemic risk assessor; and
modulate insulin delivery rates, using an insulin delivery supervisor, based on data from the comparator and from the glycemic risk assessor.

18. The system of claim 17, wherein the inputs comprise at least one of glucose data, insulin data, sensed input data, or user input data.

19. The system of claim 18, wherein identifying the discrepancies comprises quantifying the degree to which recent blood glucose measurements are inconsistent with recent insulin.

20. The system of claim 17, wherein the computer readable medium further comprises instructions that, when executed by the at least one processor, cause the system to, at the comparator:
estimate physiological or behavioral states of the patient based on received inputs, using a state estimator;
provide an output to a model agreement assessor;
for one or more state variables, evaluate discrepancies between two different models of metabolic or behavioral states; and
output the discrepancies.

21. The method of claim 20, wherein evaluating the discrepancies comprises computing a difference between a state estimator variable and what the model would have predicted absent continuous glucose monitoring (CGM) data for the same variable, the discrepancy being the difference between the two versions of the variable.

22. The method of claim 20, wherein the computer readable medium further comprises instructions that, when executed by the at least one processor, cause the system to determine an internal reference insulin rate (RIR) and output the RIR.

23. The method of claim 22, wherein the computer readable medium further comprises instructions that, when executed by the at least one processor, cause the system to, at the glycemic risk assessor:
determine a risk of at least one of current or future hyperglycemia;
determine a risk of at least one of current or future hypoglycemia; and
output the risk of at least one of current or future hyperglycemia and the risk of at least one of current or future hypoglycemia.

24. The method of claim 23, wherein the computer readable medium further comprises instructions that, when executed by the at least one processor, cause the system to, at the insulin delivery supervisor:
determine a target trajectory of future insulin;
determine an amount of insulin needed to minimize a discrepancy determined by a model agreement assessor, using a normative insulin planner;
reconcile a proposed basal rate or a proposed bolus rate with insulin needs identified by the normative insulin planner to determine an approved basal rate or an approved bolus rate; and
output the approved basal rate or the approved bolus rate.
